(19) United States
(12) Patent Application Publication
(10) Pub. No.: US 2026/0124071 A1
(43) Pub. Date: May 7, 2026

(54) SYSTEMS AND METHODS FOR DETECTION OF A VITREOUS CUTTER IN SURGICAL SYSTEM

(71) Applicant: Johnson & Johnson Surgical Vision, Inc., Irvine, CA (US)

(72) Inventors: Deep Mehta, Irvine, CA (US); Jacqueline Villalobos, Chula Vista, CA (US)

(21) Appl. No.: 18/934,732

(22) Filed: Nov. 1, 2024

(51) Int. Cl. A61F 9/007; A61B 17/00; A61B 90/00; A61B 90/90

(52) U.S. Cl. CPC A61F 9/00754; A61B 2017/00199; A61B 2017/00464; A61B 2090/064; A61B 90/90; A61B 2217/005; A61B 2217/007

(57) ABSTRACT

A computer based surgical support method to determine a type of handpiece connected to a surgical system is disclosed. The method includes providing irrigation fluid from an irrigation reservoir to the handpiece via an irrigation line; providing aspiration via an aspiration line; measuring a first vacuum level associated with the aspiration line; and based on the measured vacuum value, determining the type of handpiece coupled with the surgical system.

### FIELD OF INVENTION

This invention generally relates to surgical systems used in ocular surgery and more specifically to a phacoemulsification system in ocular surgery.

### BACKGROUND

Vitrectomy surgery has been successfully employed in the treatment of certain ocular problems, such as retinal detachments, resulting from tears or holes in the retina. Vitreous removal may also be required during a phacoemulsification procedure when the posterior capsule is ruptured causing vitreous material to move into the anterior chamber of the eye. Prior to an intraocular lens being inserted to replace the emulsified natural lens, the vitreous material must be removed. A vitreous cutting device may be used for this purpose.

Current vitreous cutting devices may employ a “guillotine” type action wherein a sharp-ended inner rigid cutting tube moves axially inside an outer sheathing tube. When the sharp-ended inner tube moves past the forward edge of a side port opening in the outer sheathing tube, the eye material (e.g., vitreous gel or fibers) is cleaved into sections small enough to be removed through the hollow center of the inner cutting tube. Vitreous cutters are available in either electric or pneumatic form. Today's electric cutters may operate within a range of speeds typically between 750-2500 cuts-per-minute (CPM) where pneumatic cutters may operate over a range of speeds between 50-5000 CPM. The surgeon may adjust control of the pneumatic vitrectomy surgical instrument cutting speed, i.e., controlling the cutting device within the handpiece, in order to perform different activities during the corrective procedure.

The cutting device within a pneumatic handpiece requires precise control of applied pressure to overcome the internal spring return mechanism to assure the quality of each cutting stroke. Today's systems typically employ a constant opening signal time to open the valve at low cutting speeds. As the selected cutting speed increases, reducing the amount of time the valve is opened is often necessary to prevent constant over-pressurizing of the handpiece at the forward end of the cutting stroke. The frequency of opening and closing the pneumatic valve, i.e., the time interval between each opening cycle of the valve, is varied to achieve the desired cutting speed.

Although most designs use variable valve opening timing and variable timing between valve openings for pneumatic vitrectomy cutter control, certain advanced designs vary the input pneumatic supply pressure as vitrectomy cutter speed changes. Such operation can enhance the quality and efficiency of material processed by the vitrectomy cutter during each cut cycle. The fundamental limitation of a variable input supply pressure vitrectomy cutter control is the shortest amount of time that the air volume in the cutter body and the associated tube set may be pressurized to reach the minimum peak pressure required to advance the cutter to a cut position and then vent to reach the minimum residual pressure to allow the spring-loaded cutter to return to a retracted position.

Further, current vitrectomy systems typically compensate for mechanical delays by providing excess pressure to extend the cutter and/or allocating excess time to retract the cutter. This type of operation is based on historical performance and some conjecture that the present situation is similar to past situations. Such operation and use of power and/or timing buffers are not optimal. Further, a certain amount of material is typically brought into the cutter based on the aspiration rate and the amount of time the cutter is open or closed, which is related to the pressure supplied to the cutter during each cut cycle. Such designs cut based on scheduled timing, resulting in more or less material cut than desired.

Today's vitrectomy surgical systems require a wide range of selectable cutting speeds and highly accurate control of the amount of pressure supplied is desirable to ensure proper instrument handpiece control and safe use in an operating theater. It may be beneficial in certain circumstances to offer the surgeon enhanced accuracy in cutting speeds, cutting efficiency, controllability, and other attributes related to performance of the vitrectomy procedure. Further, in certain circumstances benefits may be obtained by adjusting operation based on conditions encountered rather than establishing and employing operational parameters irrespective of such conditions, including altering operational parameters such as cut rate, amount of material cut, and other critical vitrectomy parameters.

Some phacoemulsification systems provide a capability to perform anterior segment vitrectomy in addition to phacoemulsification and diathermy/coagulation functions. The anterior system vitrectomy is performed using either a 20, 23 or 25 Ga cutter. The vitrectomy cutter is driven using specified pneumatic pressure to extend the cutter to close, the control algorithm then relieves the pressure to allow the built-in spring to retract the cutter to open position.

The pneumatically driven vitrectomy cutters come in single edge and dual or two edge (blade) configurations. The dual blade allows cutting in both direction of the cutter movement (open to close and close to open). Existing systems do not have a capability to detect type of cutter (single blade or dual blade) connected to the system other than RFID or QR code scanning. The capability to detect type of cutter is connected to the system without additional user input allows the system and control algorithms to configure the system settings for the connected blade and eliminates additional workflow steps for the OR staff (e.g., manually enter the type of cutter, programming the cutter setpoints for the connected cutter type or scanning an identification tag. Allowing system to configure the appropriate settings per blade type helps staff with setup and reduces setup and setting configuration errors during the procedure.

### BRIEF DESCRIPTION OF THE DRAWING(S)

The following detailed description should be read with reference to the drawings, in which like elements in different drawings are identically numbered. The drawings, which are not necessarily to scale, depict selected examples and are not intended to limit the scope of the invention. The detailed description illustrates by way of example, not by way of limitation, the principles of the invention. This description will clearly enable one skilled in the art to make and use the invention, and describes several examples, adaptations, variations, alternatives and uses of the invention, including what is presently believed to be the best mode of carrying out the invention.

FIG. 1 illustrates an exemplary phacoemulsification system in a functional block diagram;

FIG. 2 is an exemplary vitrectomy probe;

FIG. 3A illustrates an exemplary operation of a single blade vitrectomy cutter;

FIG. 3B illustrates an exemplary operation of a dual blade vitrectomy cutter;

FIG. 4 is a flow chart of an exemplary computer-based vitrectomy cutter detection algorithm;

FIG. 5 is a flow chart of an exemplary computer-based blade type evaluation algorithm;

FIG. 6 is a graph illustrating vacuum data outputted from an exemplary computer based vitrectomy cutter detection algorithm;

FIGS. 7A-7F are graphs that illustrate a single blade cutter connection; and

FIGS. 8A-8F are graphs that illustrate a dual blade cutter connection.

### DETAILED DESCRIPTION OF THE PREFERRED EXAMPLES

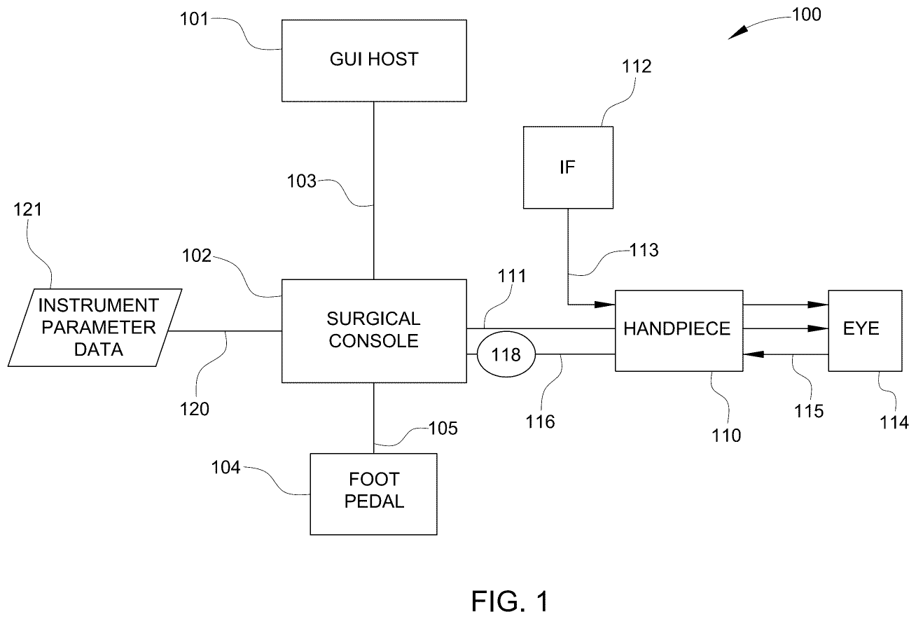

FIG. 1 illustrates an exemplary phacoemulsification system 100 in a functional block diagram that may be employed in accordance with an aspect of the present disclosure. A serial communication cable 103 connects GUI host 101 module and surgical console 102 module for the purposes of controlling the surgical console 102 by the GUI host 101. GUI host 101 and instrument host 102, as well as any other component of system 100, may be connected wirelessly. Surgical console 102 may be considered a computational device in the arrangement shown, but other arrangements are possible. An interface communications cable 120 is connected to surgical console 102 module for distributing instrument parameter/sensor data 121, and may include distribution of instrument settings and parameters information, to other systems, subsystems and modules within and external to surgical console 102 module. Although shown connected to the surgical console 102 module, interface communications cable 120 may be connected or realized on any other subsystem (not shown) that could accommodate such an interface device able to distribute the respective data.

A switch module associated with a foot pedal 104 may transmit control signals relating internal physical and virtual switch position information as input to the surgical console 102 over serial communications cable 105. The foot pedal 104 may be connected wirelessly (e.g., Bluetooth, infrared, etc. to the surgical console 102. Surgical console 102 may provide a database file system for storing configuration parameter values, programs, and other data saved in a storage device (not shown). In addition, the database file system may be realized on the GUI host 101 or any other subsystem (not shown) that could accommodate such a file system.

The phacoemulsification system 100 has a handpiece 110 that includes a needle and electrical means, typically a piezoelectric crystal, for ultrasonically vibrating the needle. The surgical console 102 supplies ultrasound power 111 to the handpiece 110. An irrigation fluid source 112 can be fluidly coupled with/to handpiece 110 through irrigation line 113 via a sleeve (not shown) that at least partially surrounds the needle and includes at least one port for delivery of the irrigation fluid by the handpiece 110 to an eye, or affected area or region, indicated diagrammatically by block 114. Alternatively, the irrigation source may be routed to eye 114 through a separate pathway independent of the handpiece 110 using a bimanual technique known in the art. Irrigation fluid may be delivered to the eye 114 by a gravity fed irrigation fluid source 112 and/or a pump fluidly coupled with the irrigation fluid source 112. The surgical console 102 controls one or more pumps. One or more pumps may provide aspiration applied to the handpiece and eye through line 116 in the direction 115 away from the eye 114 and may control irrigation fluid to the eye via the handpiece 110 through irrigation line 113. A surgeon/operator may select system parameters using the handpiece, foot pedal, via the instrument host and/or GUI host, and/or by voice command.

The phacoemulsification system 100 may include a sensor system. For example, the system 100 may include at least one sensor 118 coupled anywhere along the aspiration line 116. In an example, one or more sensors may be located in the handpiece 110, the console 102, and/or coupled anywhere along the aspiration line 116. Although not shown, the system 100 may also include at least one sensor coupled anywhere along the irrigation line 113 and/or in the handpiece 110. Measurements and/or data from the at least one sensor 118 and/or the at least one irrigation sensor may be communicated to the surgical console 102.

The surgical console 102 generally comprises at least one processor board. Surgical console 102 may include many of the components of a personal computer, such as a data bus, a memory, input and/or output devices (including a touch screen (not shown)), and the like. Surgical console 102 will often include both hardware and software, with the software typically comprising machine readable code or programming instructions for implementing one, some, or all of the methods described herein. The code may be embodied by a tangible media such as a memory, a magnetic recording media, an optical recording media, or the like.

A controller (not shown) may have (or be coupled with/to) a recording media reader, or the code may be transmitted to surgical console 102 by a network connection such as an internet, an intranet, an Ethernet, a wireless network, or the like. Along with programming code, instrument host 102 may include stored data for implementing the methods described herein, and may generate and/or store data that records parameters reflecting the treatment of one or more patients.

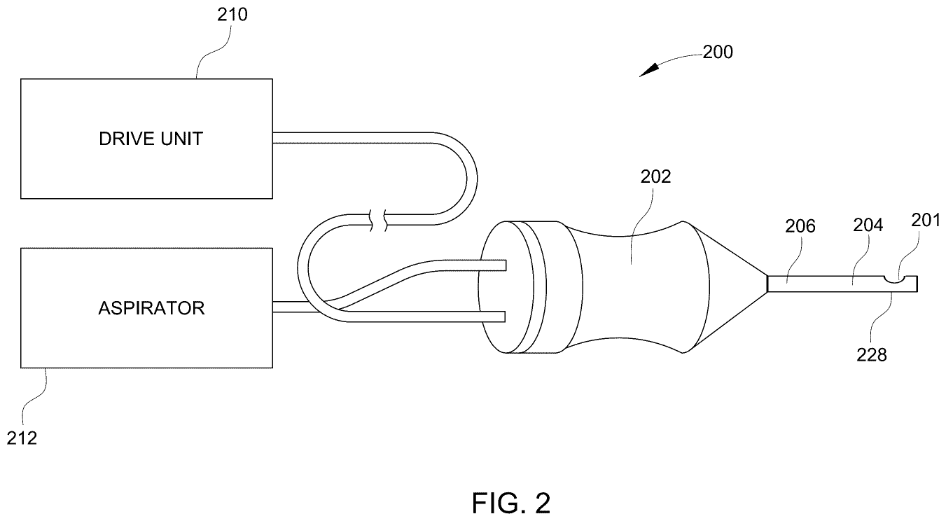

The system 100 shown in FIG. 1 may also include a vitreous cutter that is connected to system 100 in place of handpiece 110. FIG. 2 illustrates an example vitreous cutter 200 for use in ophthalmic surgery. The illustrated apparatus 200 includes a handle 202 and a vitrectomy probe 204. The vitrectomy probe 204, has a first end 206 coupled with the handle 202 and a second end 228 opposite the first end 206. The second end 228 includes port 201 for aspirating vitreous material. The vitreous cutter 200 also includes a drive unit 210 and an aspirator 212 (shown schematically in FIG. 1) coupled with handle 202. The aspirator 212 may be one or more pumps. The drive unit 210 is connected to the vitreous cutter 200 to reciprocate an internal guillotine style cutter/blade (not shown) located within the lumen of the vitrectomy probe 204. The aspirator 212 is fluidly coupled with the vitrectomy probe 204 to create a suction and remove cut pieces of tissue. The vitreous cutter may be a single blade or a dual blade vitreous cutter.

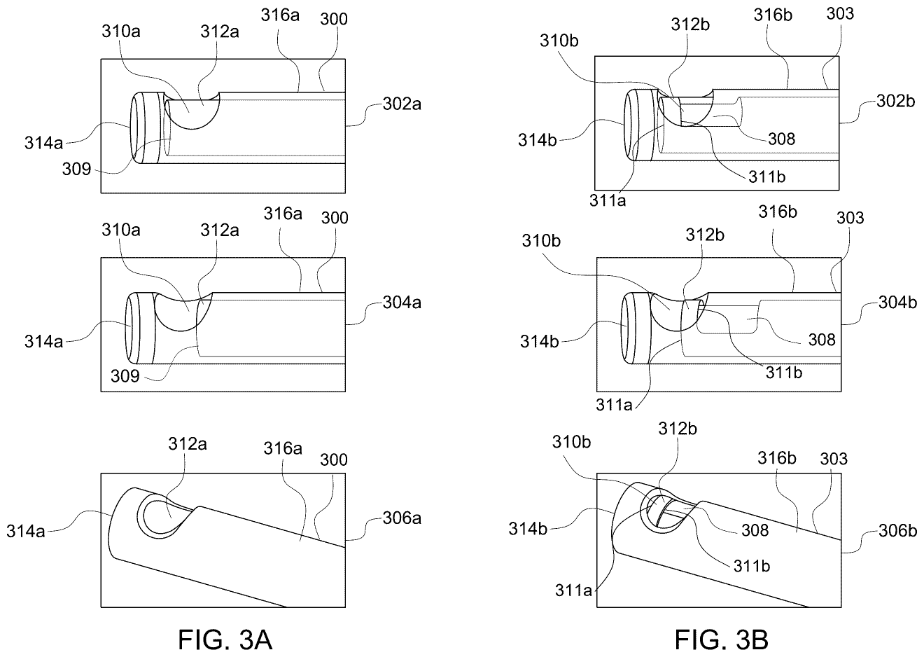

FIG. 3A illustrates the operational steps of a single blade vitreous cutter 300. As shown in 302a, when the single blade is actuated and the internal cutting tube 312a with cutting blade 309 is extended to the distal end 314a of the sheathing tube 316a, there is a complete blockage of the aspiration port 310a. This blockage prevents the aspiration flow from reaching a maximum aspiration set point of 10 cc and getting above an aspiration threshold. The maximum aspiration set point may be based on the maximum allowable aspiration allowed by the gauge type for the attached vitrector. As the measured vacuum is inversely related to the aspiration flow, the system reaches maximum vacuum. In position 304a, the internal cutting tube 312a with cutting blade 309 moves back into the sheathing tube 316a (is retracted) thereby opening the aspiration port 310a allowing additional vitreous material to enter the aspiration port 310a and exposing the cutting blade 309. In position 306a, the internal cutting tube 312a of the single blade vitreous cutter is again actuated and in a cut position as shown in 302a.

FIG. 3B illustrates the operational steps of a dual blade vitreous cutter 303. The inner rigid cutting tube 312b of vitreous cutter 303 has a distal cutting edge 311a and a proximal cutting edge 311b. In position 302b, the dual blade aspiration port 310b is unblocked or open when the inner rigid cutting tube 312b is extended to the distal end 314b of the sheathing tube 316b (similarly to when the inner rigid cutting tube is fully retracted). When the aspiration port 310b is open, the aspiration will be able to reach the commanded value and can be found above the aspiration threshold. The measured vacuum, therefore, is a smaller negative value than the value obtained with the single blade cutter 300.

As shown in FIG. 3B, the aspiration port 310b of sheathing tube 316b is unblocked or open when the internal cutting tube 312b is extended to the distal end 314b of the sheathing tube 316b due to port 308 of the internal cutting tube 312b. When aspiration port 310b is open, the aspiration will be able to reach the commanded value and can be found above the aspiration threshold. The measured vacuum, therefore, is a smaller negative value than the value obtained with the single blade cutter 300. At 304b, the cutting blade 312b is in a second cut position, wherein the cut occurs at the proximal edge 311b of the internal cutting tube 312b and aspiration port 310b and fluid and/or material is aspirated through aspiration port 310b and the distal end of the internal cutting tube 312b. At 306b, the dual cutter 303 is moving in between its position at 302b and 304b. As shown in FIG. 3B, the dual blade cutter 303, unlike the single blade cutter 300, performs two cuts (302b and 304b) when the inner cutting tube 312b is moved in a first direction (distal or proximal) and a second direction opposite the first direction (distal or proximal).

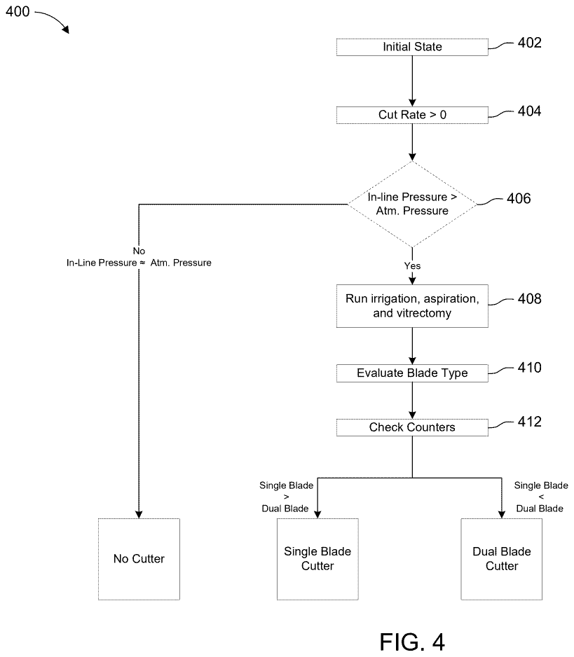

FIG. 4 is a flow chart of an exemplary computer-based vitrectomy cutter detection algorithm 400. At step 402, the system 100 has been powered on and is ready to begin the prime procedure. This may be referred to as the initial state. During the initial state, the system 100 may perform a pneumatic interface check to confirm that all pneumatic lines are connected to the system 100.

At step 404, the system 100 begins the prime procedure, starts the cutter, and sets the cut rate to a value greater than zero.

At optional step 406, the system 100 performs an in-line pressure check by comparing the in-line pressure sensor reading of the cutter to the atmospheric pressure of the environment. The inline pressure may be measured in air or the pneumatic line of the handpiece. The in-line pressure sensor may be located anywhere in the system from the handpiece to the console, e.g., at a fluid dynamic cartridge or vacuum surge prevention module coupled with the aspiration line (as described in U.S. Pat. No. 11,771,818).

Based on the reading from the in-line pressure sensor check at optional step 406, the system 100 determines whether a cutter is connected to the system or not. The in-line pressure sensor may be located near the port (i.e., cut valve).

If, at optional step 406, the in-line pressure read by the pressure sensor is greater than the atmospheric pressure and the in-line pressure is pressurized for at least the cut rate set at step 404, the system 100 determines that a cutter is connected and proceeds to step 408. If, at optional step 406, the in-line pressure read by the pressure sensor is approximately equal to atmospheric pressure, the system 100 determines that no cutter is connected to the system.

If the system 100 does not perform optional step 406, it may go directly from step 404 to step 408. At step 408, the system 100 runs both irrigation and aspiration and then reads the aspiration and vacuum values. The aspiration and vacuum thresholds that are being compared are a result of the differences between single blade and dual blade cutter position during actuation.

At step 410, the system 100 evaluates the blade type. This evaluation is described in further detail in FIG. 5.

At step 412, the system 100 analyzes counters to determine whether the cutter is a single blade cutter or dual blade cutter. The counters are described in more detail below.

FIG. 5 is a flow chart of an exemplary algorithm 500 for evaluating the blade type at step 412 as described in FIG. 4 above. The evaluation of the blade type may be continuous, such that the system 100 is continuously measuring the aspiration and vacuum value.

At step 502, the system 100 calculates aspiration and/or vacuum values If the measured aspiration level is below an aspiration threshold, and/or the vacuum measurement is above a threshold, the system 100 determines, at step 504, that a single blade cutter is connected to the system 100. Conversely, if the measured aspiration level is above a threshold, and/or the vacuum measurement is below a threshold, the system 100 determines, at step 506, that a dual blade cutter is connected to the system 100. The threshold may be determined from empirical test data. With respect to fluid flow, a single blade cutter occludes or blocks the flow of fluid each time the cutter is moved to a close position. In a dual blade cutter, the cutter is always open to fluid flow. Without occlusion, the system 100 may reach the running vacuum and with occlusion, the system reaches maximum vacuum allowed. Running vacuum is significantly lower than the maximum vacuum.

Alternatively, at step 502, the system 100 may calculate aspiration and/or vacuum values while the cutter is in a closed position (blocking the aspiration port for the single blade while the dual blade will not block or only partially block the aspiration port whether activated or not - no closed position). In a first example, for aspiration flow rate and measured vacuum, the single blade flow rate would go to zero when the blade is actuated or extended and vacuum would reach or be closer to a maximum vacuum set point. In a dual-blade cutter, the flow rate would be less than or equal to the commanded flow rate (but greater than zero) and the measured vacuum would not reach the maximum vacuum set point (less than maximum vacuum, but closer to running vacuum). If the measured aspiration level is below an aspiration threshold, and the vacuum measurement is above a threshold, the system 100 determines, at step 504, that a single blade cutter is connected to the system 100. Conversely, if the measured aspiration level is above a threshold, and the vacuum measurement is below a threshold, the system 100 determines, at step 506, that a dual blade cutter is connected to the system 100. In a second example, for measured vacuum only, single blade measured vacuum would be closer to the maximum vacuum set point and the dual blade measured vacuum would be less than the maximum vacuum setpoint. If the measured vacuum measurement is above a threshold, the system 100 determines, at step 504, that a single blade cutter is connected to the system 100. Conversely, if the measured vacuum is below or equal to a threshold, the system 100 determines, at step 506, that a dual blade cutter is connected to the system 100.

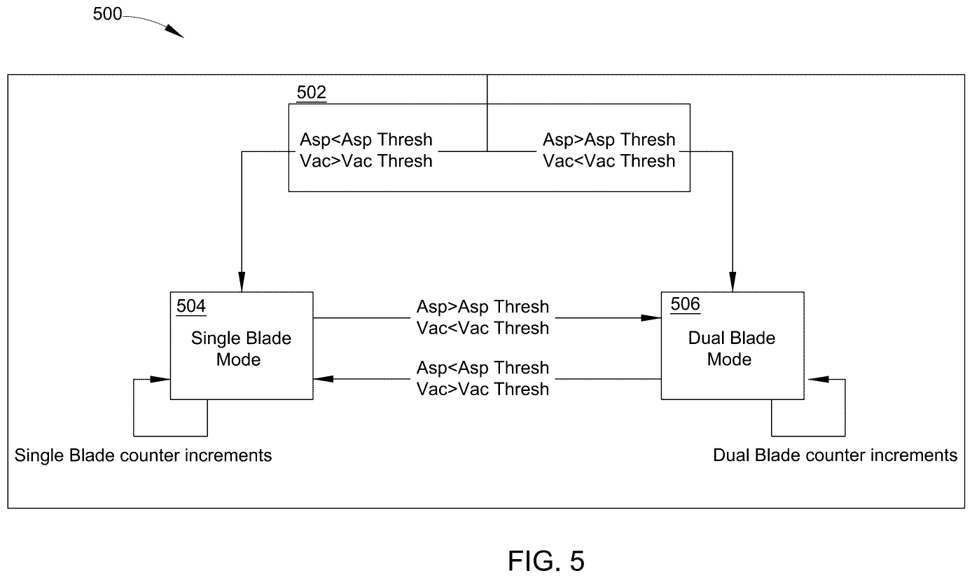

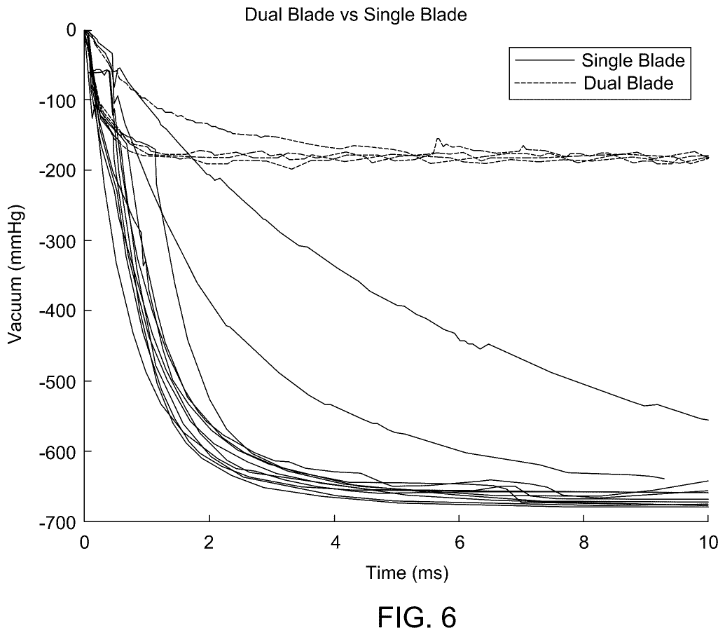

FIG. 6 is a graph illustrating vacuum data outputted from an exemplary computer based vitrectomy cutter detection algorithm. FIG. 6 illustrates an example of where only vacuum is used to determine the blade type. When the blade is actuated, the aspiration is set to 10 cc/min and vacuum is set at 700 mmHg. If single blade cutter is actuated, the port is completely blocked and the commanded aspiration causes the vacuum to increase. In contrast, if a dual blade cutter is actuated, the port remains unblocked. This allows for vacuum to remain relatively low. As a result, a vacuum threshold can be used to differentiate between these two instances and determine the connected blade type.

If the aspiration measurement and/or vacuum measurement is equal to the threshold, the system 100 may determine that a single blade cutter is connected. When a single blade is closed, the port is fully occluded the aspiration vacuum reaches a maximum vacuum setpoint and therefore, there is no aspiration flow. Based on this, the system 100 may determine that a single blade cutter is connected.

Regardless of whether the system 100 detects a single blade cutter or a dual blade cutter, the system may, optionally, increment a counter to re-check the cut rate, aspiration levels, and vacuum levels. For example, the system 100 may take an aspiration measurement and vacuum measurement every 20 milliseconds over a period of 5 to 10 seconds, meaning that the system may take between 250 and 500 measurements. After each measurement, the system 100 may determine whether a single blade cutter or a dual blade cutter is connected and update the appropriate counter. For example, if the system 100 determines, after a measurement, that a single blade cutter is connected, it may update the single blade counter by a value of 1. Similarly, if the system 100 determines, after a measurement, that a dual blade cutter is connected, it may update the dual blade counter by a value of 1.

Referring back to FIG. 4, at step 414, the system 100 may check the counters to make a final determination as to whether a single blade cutter or dual-blade cutter is connected to the system. If the counter counts more single blades than dual blades within a selected time frame (i.e., the aspiration level is below an aspiration threshold and/or the vacuum measurement is above a threshold), the system determines that a single blade cutter is connected. If the counter counted more dual blades than single blades within the selected time frame (i.e., the aspiration level is above a threshold, and/or the vacuum measurement is below a threshold, the system determines that a dual blade cutter is connected. However, as indicated above, the use of the counter to determine the type of cutter is optional. The system 100 may take a single measurement and determine the cutter type.

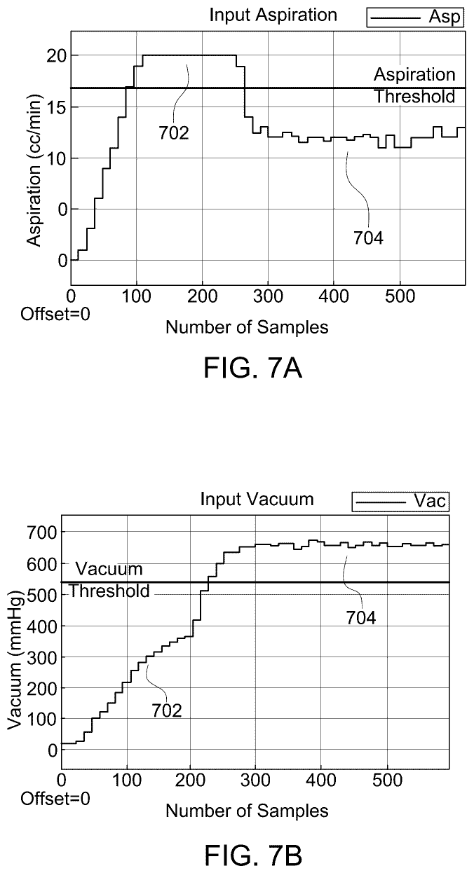

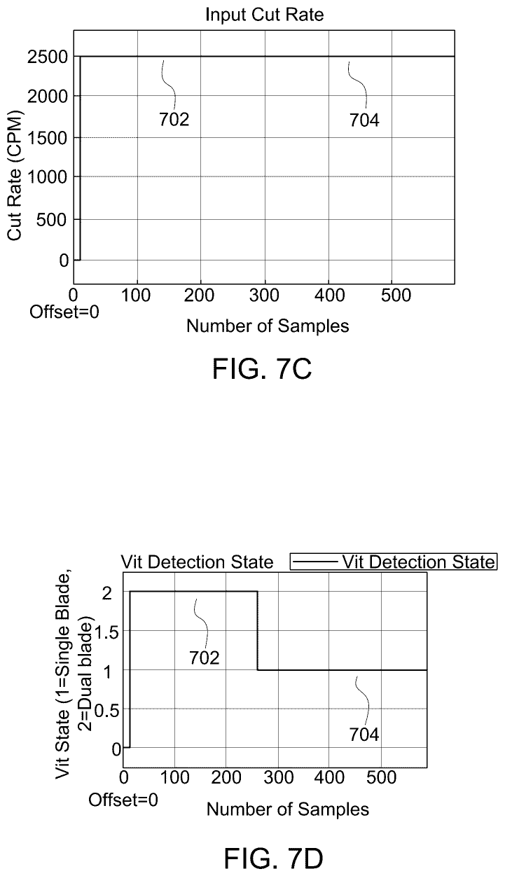

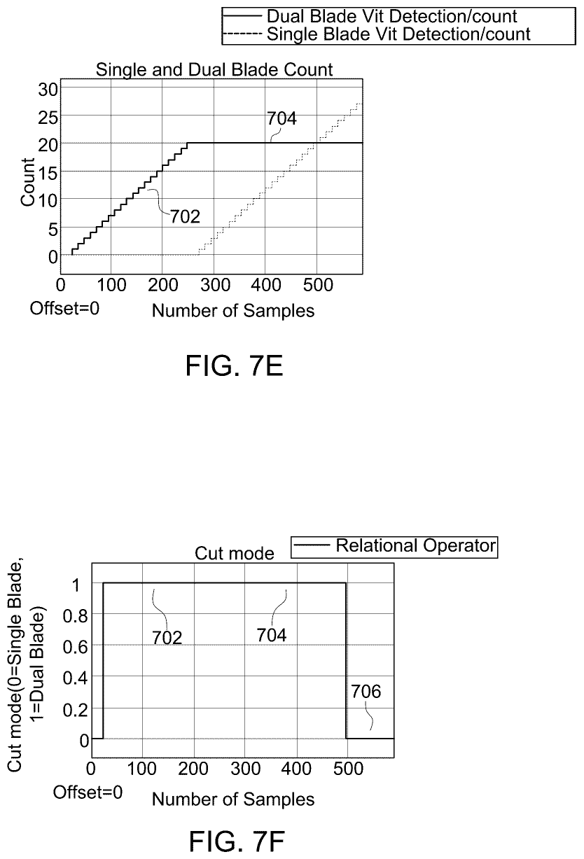

FIGS. 7A to 7F are graphs that represent the aspiration and vacuum measurements recorded during a prime procedure when a single blade cutter is connected to the system. FIG. 7A depicts an exemplary aspiration flow rate, FIG. 7B depicts an exemplary vacuum pressure, FIG. 7C depicts an exemplary cut rate, FIG. 7D represents detection states, FIG. 7E represents single blade and dual blade counts, and FIG. 7F represents the cut mode (0 represents a single blade cutter and 1 represents a dual blade cutter).

In FIG. 7A, the x-axis represents the number of samples collected, e.g., over a period of time such as 12 seconds with a sample taken every 20 milliseconds (ms), and the y-axis represents the measured aspiration in cc/min. In FIG. 7B, the x-axis represents time and the y-axis represents the measured vacuum in mmHg. In FIG. 7C, the x-axis represents the number of samples collected, e.g., over a period of time such as 12 seconds with a sample taken every 20 ms, and the y-axis represents the measured cut rate in cut rate per minute (CPM). In FIG. 7D, the x-axis represents the number of samples collected, e.g., over a period of time such as 12 seconds with a sample taken every 20 ms, and the y-axis represents the vit state, where a value of 1 represents a single blade and a value of 2 represents a dual blade. In FIG. 7E, the x-axis represents the number of samples collected, e.g., over a period of time such as 12 seconds with a sample taken every 20 ms, and the y-axis represents the single and dual blade count (i.e., how many time the system 100 detected a single blade and how many times the system detected a dual blade). In FIG. 7F, the x-axis represents the number of samples collected, e.g., over a period of time such as 12 seconds with a sample taken every 20 ms, and the y-axis represents the cut mode (i.e., single blade or dual blade). In an example, other time periods and sampling times are envisioned.

As represented in the graphs, the measurements were taken when a 23 Ga single blade cutter was connected the system. As shown in FIG. 7C, the blade was run at 2500 cuts per minute (CPM). When the single blade is actuated, the aspiration port becomes fully blocked. The repetitive blocking of the aspiration port by the cutter prevents the aspiration from stabilizing at a set point of 20 cc/min (see FIG. 7A). Because vacuum is inversely proportional to aspiration, as aspiration is unable to reach its set point, vacuum increases (see FIG. 7B).

When the cut rate is greater than zero, the algorithm described above in FIG. 5 begins to check whether a single blade cutter or a dual blade cutter is connected to the system. As shown in FIG. 7A and FIG. 7B, when a single blade cutter is connected to the system, both aspiration and vacuum begin to build.

When the first parameters settle (i.e., the system 100 reaches the maximum flow rate it can support) at time 702, the aspiration is approximately 20 cc/min and the vacuum is approximately 300 mmHg. These values match dual blade conditions by having an aspiration above the threshold and a vacuum below the threshold. Accordingly, the system 100 initially determines that a dual blade cutter is connected and increments the dual blade counter.

At time 704, after the parameters stabilize, aspiration and vacuum measurement values settle to new values based on the aspiration port being blocked when the single blade cutter is fully actuated. These values fall below the aspiration threshold and above the vacuum threshold. This matches the conditions for single blade and the state flow transitions to single-blade mode and the single blade counter increments.

FIG. 7D represents the detection state. A value of one represents a single blade cutter and a value of two represents a dual blade cutter. At time 702, the system detects a dual blade cutter and at time 704, the system detects a single blade cutter.

As shown in FIG. 7E, the system increments either the single mode cutter counter or dual blade cutter depending on the cutter the system detects. Therefore, the solid line represents the counter for dual blade mode and the dashed line represents the single blade counter. As shown in FIGS. 7D and 7E, at first the dual blade counter increments, however, as the system begins to detect a single blade cutter, the dual blade counter stops incrementing and the single blade counter begins to increment.

In FIG. 7F, the counter that is larger, as shown in FIG. 7E, defines the predicted state. A state of one indicates that the predicted state is dual blade and a state of zero indicates the predicted state is single blade. As shown in FIG. 7E, at time=500, the value of the single blade count becomes higher than the value of the dual blade account. Accordingly, the final predicted state, shown at 706 in FIG. 7F, indicates a single blade probe is attached based on the counter for the single blade being higher and the predicted state ending at zero.

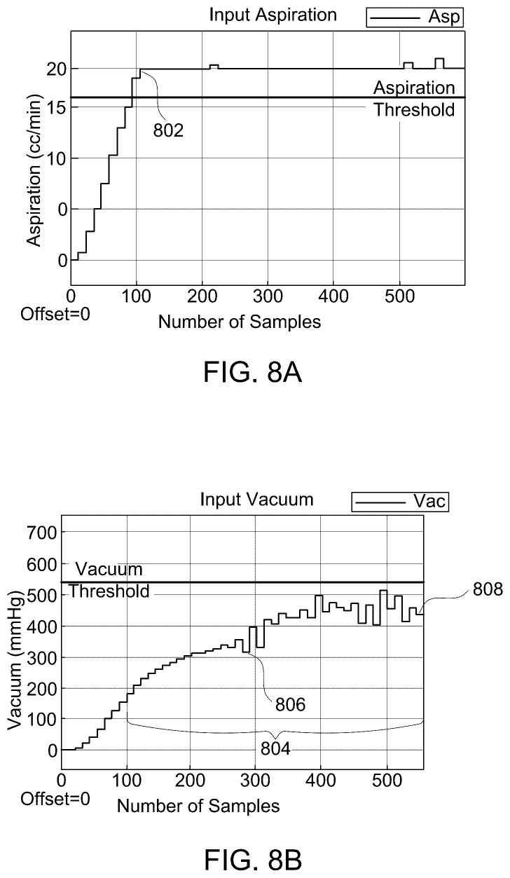

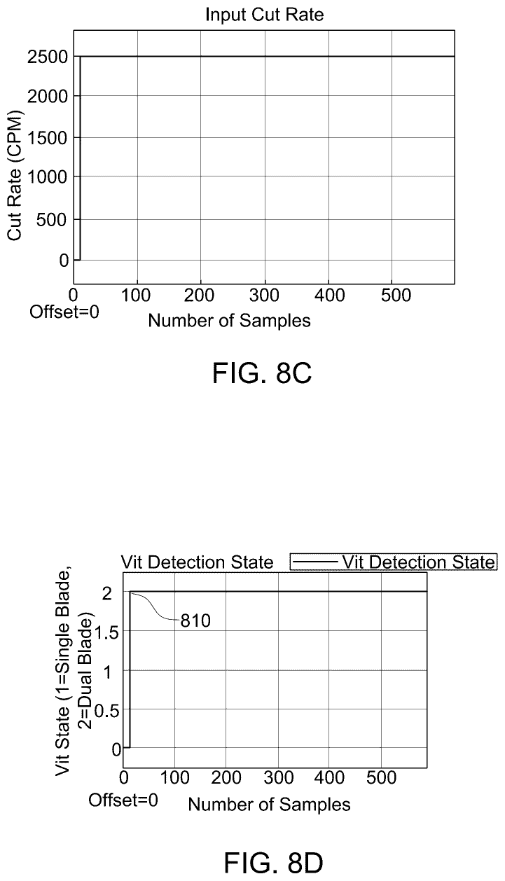

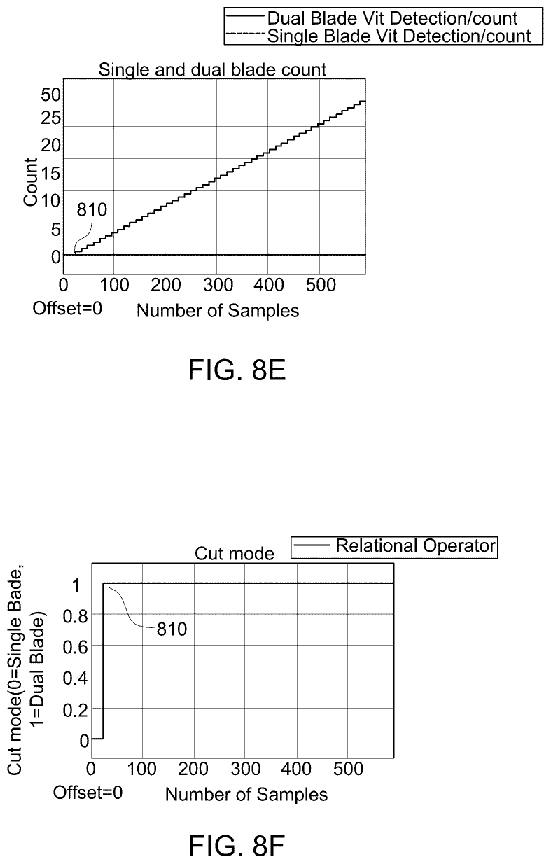

FIGS. 8A to 8F are graphs that represent the aspiration and vacuum measurements recorded during a prime procedure when a dual blade cutter is connected to the system. FIG. 8A depicts an exemplary aspiration flow rate, FIG. 8B depicts an exemplary vacuum pressure, FIG. 8C depicts an exemplary cut rate, FIG. 8D represents detection states, FIG. 8E represents single blade and dual blade counts, and FIG. 8F represents the cut mode (0 represents a single blade cutter and 1 represents a dual blade cutter).

In FIG. 8A, the x-axis represents the number of samples collected, e.g., over a period of time such as 12 seconds with a sample taken every 20 ms, and the y-axis represents the measured aspiration in cc/min. In FIG. 8B, the x-axis represents the number of samples collected, e.g., over a period of time such as 12 seconds with a sample taken every 20 ms, and the y-axis represents the measured vacuum in mmHg. In FIG. 8C, the x-axis represents the number of samples collected, e.g., over a period of time such as 12 seconds with a sample taken every 20 ms, and the y-axis represents the measured cut rate in cut rate per minute (CPM). In FIG. 8D, the x-axis represents the number of samples collected, e.g., over a period of time such as 12 seconds with a sample taken every 20 ms, and the y-axis represents the vit state, where a value of 1 represents a single blade and a value of 2 represents a dual blade. In FIG. 8E, the x-axis represents the number of samples collected, e.g., over a period of time such as 12 seconds with a sample taken every 20 ms, and the y-axis represents the single and dual blade count (i.e., how many time the system 100 detected a single blade and how many times the system detected a dual blade). In FIG. 8F, the x-axis represents the number of samples collected, e.g., over a period of time such as 12 seconds with a sample taken every 20 ms, and the y-axis represents the cut mode (i.e., single blade or dual blade). In an example, other time periods and sampling times are envisioned.

As explained above, when a dual blade cutter is connected to the system 100 and the blade is either fully actuated or unactuated, the aspiration port remains unblocked. The small blade minimally obstructs the aspiration port when the cutter is in motion, but does not obstruct the aspiration port when the blade is fully actuated or unactuated. As shown in FIG. 8A, this allows the aspiration to increase to a commanded value of 20 cc/min at time 802. Because aspiration and vacuum are inversely proportional, less vacuum is built up with a dual blade cutter than with a single blade cutter. As shown in FIG. 8B, at time 806, the vacuum reaches a steady state value that is lower than the predefined vacuum threshold.

When the cut rate is greater than zero, the algorithm described in FIG. 5 begins to check whether a single blade or dual blade is connected to the system. As shown in FIG. 8A and FIG. 8B, when the cutter and fluidics begin, aspiration and vacuum begin to build.

As shown in FIG. 8A, when the first parameters settle at time 802 (i.e., 100 ms), aspiration is about 20 cc/min. As shown in FIG. 8B, when the aspiration reaches 20 cc/min (as shown in FIG. 8A), the vacuum continues to rise during the time duration 804. During the time duration 804, the vacuum eventually settles to approximately 420 mmHg at time 808. These measurement values match dual blade conditions because the aspiration measurement is above the threshold and the vacuum measurement is below the threshold. The system 100 determines that a dual blade cutter is connected mode and increments the dual blade counter.

FIG. 8C represents the measured cut rate in CPM. As shown in FIG. 8C, the blade was run at 2500 CPM.

FIG. 8D represents the detection state. A value of one represents a single blade cutter and a value of two represents a dual blade cutter. At time 810 and forward, the system detects a dual blade cutter.

As shown in FIG. 8E, the system 100 increments either the single mode cutter counter or dual blade cutter depending on the cutter the system detects. Therefore, the solid line represents the counter for dual blade mode and the dashed line represents the single blade counter. As shown in FIG. 8E, because the system detects a dual blade cutter starting at time 810, the dual blade counter increments and the single blade counter stays at zero and does not increment.

In FIG. 8F, the counter that is larger defines the predicted state. A state of one indicates that the predicted state is dual blade cutter and a state of zero indicates the predicted state is single blade cutter. At time 810, the counter for a dual blade cutter is higher than a single blade cutter. Because the counter in FIG. 8E continues to detect a dual blade cutter, the system never determines that a single blade cutter is connected to the system 100. The final predicted state indicates that a dual blade cutter is attached.

### EXAMPLES

### Example 1

A computer based surgical support method to determine a type of handpiece (110) connected to a surgical system (100), the method comprising: providing aspiration via an aspiration line (116); measuring a first vacuum associated with the aspiration line (116); and based on the measured vacuum value, determining the type of handpiece (110) coupled with the surgical system (100).

### Example 2

The method of example 1, further comprising: measuring one or more additional vacuums associated with the aspiration line (116) and after each measurement; determining the type of handpiece (110) coupled with the surgical system (100); and based on the determined type of handpiece (110), updating a counter associated with the type of handpiece (110).

### Example 3

The method of example 2, wherein the determined type of handpiece (110) is selected from the group consisting of a single blade cutter (300) and a dual blade cutter (303).

### Example 4

The method of any of examples 1-3, wherein on a condition that the measured vacuum is above a predetermined vacuum threshold, determining that the handpiece (110) is a single blade cutter (300).

### Example 5

The method of any of examples 1-4, wherein on a condition that the measured vacuum is below a predetermined vacuum threshold, determining that the handpiece (110) is a dual blade cutter (303)

### Example 6

The method of any of examples 1-5, further comprising: measuring a first aspiration value associated the aspiration line (116) and based on the measured aspiration value, determining the type of handpiece (110) coupled with the surgical system (100).

### Example 7

The method of any of examples 1-6, further comprising: measuring one or more additional aspiration values associated with the aspiration line (116) and after each measurement, determining the type of handpiece (110) coupled with the surgical system (100). And based on that determination, updating a counter associated with the type of handpiece (110).

### Example 8

The method of any of examples 1-7, wherein the determined type of handpiece (110) is selected from the group consisting of a single blade cutter (300) and a dual blade cutter (303).

### Example 9

The method of any of examples 1-8, wherein on a condition that the aspiration value is below a predetermined aspiration threshold, determining that the handpiece (110) is a single blade cutter (300).

### Example 10

The method of any of examples 1-9, wherein on a condition that the aspiration value is above a predetermined aspiration threshold, determining that the handpiece (110) is a dual blade cutter (303).

### Example 11

The method of any of examples 1 to 10, further comprising: comparing an in-line pressure of the handpiece (110) to atmospheric pressure; and on a condition that the in-line pressure is substantially equal to the atmospheric pressure, determining that no handpiece (110) is connected to the surgical system (100).

### Example 12

The method of any of examples 1 to 11, wherein the handpiece (110) is a vitrectomy handpiece (200).

### Example 13

A computer based surgical support method to determine a type of handpiece (110) connected to a surgical system (100), the method comprising: providing irrigation fluid from an irrigation reservoir to the handpiece (110) via an irrigation line (113); providing aspiration via an aspiration line (116); measuring an aspiration rate and a vacuum of the aspiration line (116); and based on the measured aspiration rate and measured vacuum, determining the type of handpiece (110) connected to the surgical system (100).

### Example 14

The method of example 13, wherein, on a condition that the measured aspiration rate is below a predetermined aspiration threshold, and the measured vacuum is above a predetermined threshold, determining that the handpiece (110) is a single blade cutter (300).

### Example 15

The method of example 13, wherein, on a condition that the measured aspiration rate is above a predetermined aspiration threshold, and the measured vacuum is below a predetermined vacuum threshold, determining that the handpiece (110) is a dual blade cutter (303).

### Example 16

The method of example 13 further comprising: measuring one or more additional vacuums associated with the aspiration line (116); measuring one or more additional aspiration values associated with the aspiration line (116); after each measurement of the one or more additional vacuum levels and aspiration values, determining the type of handpiece (110) coupled with the surgical system (100); and based on that determination, updating a counter associated with the type of handpiece (110).

### Example 17

The method of example 18 wherein the determined type of handpiece (110) is selected from the group consisting of a single blade cutter (300) and a dual blade cutter (303).

### Example 18

A surgical system (100) comprising: a handpiece (110), an irrigation line (113) coupled with the handpiece (110), an aspiration line (116) coupled with the handpiece (110), a sensor communicatively coupled with the aspiration line (116), and a surgical console communicatively coupled with the handpiece (110), wherein the sensor and the surgical console are configured to: provide irrigation fluid from an irrigation reservoir to the handpiece (110) via the irrigation line (113); provide aspiration via the aspiration line (116); measure a first vacuum level associated with the aspiration line (116); and based on the measured vacuum value, determining a type of handpiece (110) coupled with the surgical system (100).

### Example 19

The surgical system (100) of example 18, wherein the sensor and surgical console are further configured to: measure one or more additional vacuum levels associated with the aspiration line (116); after each measurement of the one or more additional vacuum levels, determine the type of handpiece (110) coupled with the surgical system (100); and based on the determined type of handpiece (110), update a counter associated with the type of handpiece (110).

### Example 20

The surgical system (100) of any of examples 18-19, wherein the determined type of handpiece (110) is selected from the group consisting of a single blade cutter (300) and a dual blade cutter (303).

### Example 21

The surgical system (100) of any of examples 18-20, wherein on a condition that the measured vacuum is above a predetermined vacuum threshold, determining that the handpiece (110) is a single blade cutter (300).

### Example 22

The surgical system (100) of any of examples 18-21, wherein on a condition that the measured vacuum is below a predetermined vacuum threshold, determining that the handpiece (110) is a dual blade cutter (303)

### Example 23

The surgical system (100) of any of examples 18-22, wherein the sensor and surgical console are further configured to measure a first aspiration value associated the aspiration line (116) and based on the measured aspiration value and determine the type of handpiece (110) coupled with the surgical system (100).

### Example 24

The surgical system (100) of any of examples 18-23, wherein the sensor and surgical console are further configured to measure one or more additional aspiration values associated with the aspiration line (116) and after each measurement and determine the type of handpiece (110) coupled with the surgical system (100); and based on that determination, update a counter associated with the type of handpiece (110).

### Example 25

The surgical system (100) of any of examples 18-24, wherein the determined type of handpiece (110) is selected from the group consisting of a single blade cutter (300) and a dual blade cutter (303).

### Example 26

The surgical system (100) of any of examples 18-25, wherein on a condition that the aspiration value is below a predetermined aspiration threshold, determining that the handpiece (110) is a single blade cutter (300).

### Example 27

The surgical system (100) of any of examples 18-26, wherein on a condition that the aspiration value is above a predetermined aspiration threshold, determining that the handpiece (110) is a dual blade cutter (303).

### Example 28

The surgical system (100) of any of examples 18-27, wherein the sensor and the surgical console are further configured to: compare an in-line pressure of the handpiece (110) to atmospheric pressure; and on a condition that the in-line pressure is substantially equal to the atmospheric pressure, determine that no handpiece (110) is connected to the surgical system (100).

### Example 29

The surgical system (100) of any of examples 1-28, wherein the handpiece (110) is a vitrectomy handpiece (200).

### Example 30

A surgical system (100) comprising: a handpiece (110), an irrigation line (113) coupled with the handpiece (110), an aspiration line (116) coupled with the handpiece (110), a sensor communicatively coupled with the aspiration line (116), and a surgical console communicatively coupled with the handpiece (110), wherein the sensor and the surgical console are configured to: provide irrigation fluid from an irrigation reservoir to the handpiece (110) via an irrigation line (113); provide aspiration via an aspiration line (116); measure an aspiration rate and a vacuum of the aspiration line (116); and based on the measured aspiration rate and measured vacuum, determine the type of handpiece (110) connected to the surgical system (100).

### Example 31

The surgical system (100) of example 30, wherein, on a condition that the measured aspiration rate is below a predetermined aspiration threshold, and the measured vacuum is above a predetermined threshold, determining that the handpiece (110) is a single blade cutter (300).

### Example 32

The surgical system (100) of any of examples 30-31, wherein, on a condition that the measured aspiration rate is above a predetermined aspiration threshold, and the measured vacuum is below a predetermined vacuum threshold, determining that the handpiece (110) is a dual blade cutter (303).

### Example 33

The surgical system (100) of any of examples 30-32, wherein the sensor and surgical console are further configured to: measure one or more additional vacuums associated with the aspiration line (116); measure one or more additional aspiration values associated with the aspiration line (116); after each measurement of the one or more additional vacuum levels and aspiration values, determine the type of handpiece (110) coupled with the surgical system (100); and based on that determination, update a counter associated with the type of handpiece (110).

### Example 34

The surgical system (100) of any of examples 30-33, wherein the determined type of handpiece (110) is selected from the group consisting of a single blade cutter (300) and a dual blade cutter (303).

It should be understood that many variations are possible based on the disclosure herein. Although features and elements are described above in particular combinations, each feature or element can be used alone without other features and elements or in various combinations with or without other features and elements.

What is claimed is:

1. A computer based surgical support method to determine a type of handpiece connected to a surgical system, the method comprising:
providing irrigation fluid from an irrigation reservoir to the handpiece via an irrigation line;
providing aspiration via an aspiration line;
measuring a first vacuum level associated with the aspiration line; and
based on the measured vacuum value, determining the type of handpiece coupled with the surgical system.

2. The method of claim 1, further comprising:
measuring one or more additional vacuum levels associated with the aspiration line;
after each measurement of the one or more additional vacuum levels, determining the type of handpiece coupled with the surgical system; and
based on the determined type of handpiece, updating a counter associated with the type of handpiece.

3. The method of claim 2, wherein the determined type of handpiece is selected from the group consisting of a single blade cutter and a dual blade cutter.

4. The method of claim 1, wherein on a condition that the measured vacuum level is above a predetermined vacuum threshold, determining that the handpiece is a single blade cutter.

5. The method of claim 1, wherein on a condition that the measured vacuum level is below a predetermined vacuum threshold, determining that the handpiece is a dual blade cutter.

6. The method of claim 1, further comprising:
measuring a first aspiration value associated with the aspiration line; and
based on the measured aspiration value, determining the type of handpiece coupled with the surgical system.

7. The method of claim 6, further comprising:
measuring one or more additional aspiration values associated with the aspiration line;
after each measurement of the one or more additional aspiration values, determining the type of handpiece coupled with the surgical system; and
based on the determined type of handpiece, updating a counter associated with the type of handpiece.

8. The method of claim 7, wherein the determined type of handpiece is selected from the group consisting of a single blade cutter and a dual blade cutter.

9. The method of claim 6, wherein on a condition that the aspiration value is below a predetermined aspiration threshold, determining that the handpiece is a single blade cutter.

10. The method of claim 6, wherein on a condition that the aspiration value is above a predetermined aspiration threshold, determining that the handpiece is a dual blade cutter.

11. The method of claim 1, further comprising:
comparing an in-line pressure of the handpiece to atmospheric pressure; and
on a condition that the in-line pressure is substantially equal to the atmospheric pressure, determining that no handpiece is connected to the surgical system.

12. The method of claim 1, wherein the handpiece is a vitrectomy handpiece.

13. A computer based surgical support method to determine a type of handpiece connected to a surgical system, the method comprising:
providing irrigation fluid from an irrigation reservoir to the handpiece via an irrigation line;
providing aspiration via an aspiration line;
measuring an aspiration rate and a vacuum level of the aspiration line; and
based on the measured aspiration rate and measured vacuum level, determining the type of handpiece connected to the surgical system.

14. The method of claim 13, wherein, on a condition that the measured aspiration rate is below a predetermined aspiration threshold, and the measured vacuum level is above a predetermined vacuum threshold, determining that the handpiece is a single blade cutter.

15. The method of claim 13, wherein, on a condition that the measured aspiration rate is above a predetermined aspiration threshold, and the measured vacuum level is below a predetermined vacuum threshold, determining that the handpiece is a dual blade cutter.

16. The method of claim 13, further comprising:
measuring one or more additional vacuum levels associated with the aspiration line; and
measuring one or more additional aspiration values associated with the aspiration line;
after each measurement of the one or more additional vacuum levels and aspiration values, determining the type of handpiece coupled with the surgical system; and
based on the determined type of handpiece, updating a counter associated with the type of handpiece.

17. The method of claim 16, wherein the determined type of handpiece is selected from the group consisting of a single blade cutter and a dual blade cutter.

18. A surgical system comprising:
a handpiece;
an irrigation line coupled with the handpiece;
an aspiration line coupled with the handpiece;
a sensor communicatively coupled with the aspiration line; and
a surgical console communicatively coupled with the handpiece;
wherein the sensor and the surgical console are configured to:
provide irrigation fluid from an irrigation reservoir to the handpiece via the irrigation line;
provide aspiration via the aspiration line;
measure a first vacuum level associated with the aspiration line; and
based on the measured vacuum value, determining a type of handpiece coupled with the surgical system.

19. The surgical system of claim 18, wherein the sensor and surgical console are further configured to:
measure one or more additional vacuum levels associated with the aspiration line;
after each measurement of the one or more additional vacuum levels, determine the type of handpiece coupled with the surgical system; and
based on the determined type of handpiece, update a counter associated with the type of handpiece.

20. The surgical system of claim 19, wherein the determined type of handpiece is selected from the group consisting of a single blade cutter and a dual blade cutter.

21. The surgical system of claim 18, wherein on a condition that the measured vacuum level is above a predetermined vacuum threshold, determining that the handpiece is a single blade cutter.

22. The surgical system of claim 18, wherein on a condition that the measured vacuum level is below a predetermined vacuum threshold, determining that the handpiece is a dual blade cutter.

23. The surgical system of claim 18, wherein the sensor and surgical console are further configured to:
measure a first aspiration value associated with the aspiration line; and
determine the type of handpiece couple with the surgical system.

24. The surgical system of claim 23, wherein the sensor and surgical console are further configured to:
measure one or more additional aspiration values associated with the aspiration line;
after each measurement of the one or more additional aspiration values, determine the type of handpiece coupled with the surgical system; and
based on the determined type of handpiece, update a counter associated with the type of handpiece.

25. The surgical system of claim 24, wherein the determined type of handpiece is selected from the group consisting of a single blade cutter and a dual blade cutter.

26. The surgical system of claim 23, wherein on a condition that the aspiration value is below a predetermined aspiration threshold, determining that the handpiece is a single blade cutter.

27. The surgical system of claim 23, wherein on a condition that the aspiration value is above a predetermined aspiration threshold, determining that the handpiece is a dual blade cutter.

28. The surgical system of claim 18, wherein the sensor and the surgical console are further configured to:
compare an in-line pressure of the handpiece to atmospheric pressure; and
on a condition that the in-line pressure is substantially equal to the atmospheric pressure, determine that no handpiece is connected to the surgical system.

29. The surgical system of claim 18, wherein the handpiece is a vitrectomy handpiece.

30. A surgical system comprising:
a handpiece;
an irrigation line coupled with the handpiece;
an aspiration line coupled with the handpiece;
a sensor communicatively coupled with the aspiration line; and
a surgical console communicatively coupled with the handpiece;
wherein the sensor and surgical console are configured to:
provide irrigation fluid from an irrigation reservoir to the handpiece via the irrigation line;
provide aspiration via the aspiration line;
measure an aspiration rate and a vacuum level of the aspiration line; and
based on the measured aspiration rate and measured vacuum level, determine a type of handpiece coupled with the surgical system.

31. The surgical system of claim 30, wherein, on a condition that the measured aspiration rate is below a predetermined aspiration threshold, and the measured vacuum level is above a predetermined vacuum threshold, determining that the type of handpiece is a single blade cutter.

32. The surgical system of claim 30, wherein, on a condition that the measured aspiration rate is above a predetermined aspiration threshold, and the measured vacuum level is below a predetermined vacuum threshold, determining that the type of handpiece is a dual blade cutter.

33. The surgical system of claim 30, further comprising:
measure one or more additional vacuum levels associated with the aspiration line; and
measure one or more additional aspiration values associated with the aspiration line;
after each measurement of the one or more additional vacuum levels and aspiration values, determine the type of handpiece coupled with the surgical system; and
based on the determined type of handpiece, update a counter associated with the type of handpiece.

34. The surgical system of claim 33, wherein the determined type of handpiece is selected from the group consisting of a single blade cutter and a dual blade cutter.
